SEQUENCE LISTING (18 sequences)

SEQ ID NO: 1 - protein, 714 aa, Clostridium difficile
MNKKNLAMAMAAVTVVGSAAPIFADSTTPGYTVVKNDWKKAVKQLQDGLKNKTISTIKVSFNGNSVGEVTPASSGAKKADRDAAAEKLYNLVNTQLDKLGDGDYVDFEVTYNLATQIITKAEAEAVLTKLQQYNDKVLINSATDTVKGMVSDTQVDSKNVAANPLKVSDMYTIPSAITGSDDSGYSIAKPTEKTTSLLYGTVGDATAGKAITVDTASNEAFAGNGKVIDYNKSFKATVQGDGTVKTSGVVLKDASDMAATGTIKVRVTSAKEESIDVDSSSYISAENLAKKYVFNPKEVSEAYNAIVALQNDGIESDLVQLVNGKYQVIFYPEGKRLETKSADIIADADSPAKITIKANKLKDLKDYVDDLKTYNNTYSNVVTVAGEDRIETAIELSSKYYNSDDKNAITDDAVNNIVLVGSTSIVDGLVASPLASEKTAPLLLTSKDKLDSSVKSEIKRVMNLKSDTGINTSKKVYLAGGVNSISKDVENELKNMGLKVTRLSGEDRYETSLAIADEIGLDNDKAFVVGGTGLADAMSIAPVASQLKDGDATPIVVVDGKAKEISDDAKSFLGTSDVDIIGGKNSVSKEIEESIDSATGKTPDRISGDDRQATNAEVLKEDDYFKDGEVVNYFVAKDGSTKEDQLVDALAAAPIAGRFKESPAPIILATDTLSSDQNVAVSKAVPKDGGTNLVQVGKGIASSVINKMKDLLDM

SEQ ID NO: 2 - protein, 719 aa, Clostridium difficile
MNKKNIAIAMSGLTVLASAAPVFAATTGTQGYTVVKNDWKKAVKQLQDGLKDNSIGKITVSFNDGVVGEVAPKSANKKADRDAAAEKLYNLVNTQLDKLGDGDYDDFSVDYNLENKIITNQADAEAIVTKLNSLNEKTLIDIATKDTFGMVSKTQDSEGKNVAATKALKVKDVATFGLKSGGSEDTGYVVEMKAGAVEDKYGKVGDSTAGIAINLPSTGLEYAGKGTTIDFNKTLKVDVTGGSTPSAVAVSGFVTKDDTDLAKSGTINVRVINAKEESIDIDASSYTSAENLAKRYVFDPDEISEAYKAIVALQNDGIESNLVQLVNGKYQVIFYPEGKRLETKSANDTIASQDTPAKVVIKANKLKDLKDYVDDLKTYNNTYSNVVTVAGEDRIETAIELSSKYYNSDDKNAITDKAVNDIVLVGSTSIVDGLVASPLASEKTAPLLLTSKDKLDSSVKSEIKRVMNLKSDTGINTSKKVYLAGGVNSISKDVENELKNMGLKVTRLSGEDRYETSLAIADEIGLDNDKAFVVGGTGLADAMSIAPVASQLKDGDATPIVVVDGKAKEISDDAKSFLGTSDVDIIGGKNSVSKEIEESIDSATGKTPDRISGDDRQATNAEVLKEDDYFTDGEVVNYFVAKDGSTKEDQLVDALAAAPIAGRFKESPAPIILATDTLSSDQNVAVSKAVPKDGGTNLVQVGKGIASSVINKMKDLLDM

SEQ ID NO: 3 - protein, 756 aa, Clostridium difficile
MNKKNIAIAMSGLTVLASAAPVFADDTKVETGDQGYTVVQSKYKKAVEQLQKGILDGSITEIKVFFEGTLASTIKVGSELNAADASKLLFTQVDNKLDNLGDGDYVDFLITSPGQGDKITTSKLVALKDLTGASADAIIAGTSSADGVVTNTGAASGSTETNSAGTKLAMSAIFDTAYTDSSETAVKITIKADMNDTKFGKAGETTYSTGLTFEDGSTEKIVKLGDSDIIDITKALKLTVVPGSKATVKFAEKTPSASVQPVITKLRIINAKEETIDIDASSSKTAQDLAKKYVFNKTDLNTLYKVLNGDEADTNGLIEEVSGKYQVVLYPEGKRVTTKSAAKASIADENSPVKLTLKSDKKKDLKDYVDDLRTYNNGYSNAIEVAGEDRIETAIALSQKYYNSDDENAIFRDSVDNVVLVGGNAIVDGLVASPLASEKKAPLLLTSKDKLDSSVKAEIKRVMNIKSTTGINTSKKVYLAGGVNSISKEVENELKDMGLKVTRLAGDDRYETSLKIADEVGLDNDKAFVVGGTGLADAMSIAPVASQLRNANGKMDLADGDATPIVVVDGKAKTINDDVKDFLDDSQVDIIGGENSVSKDVENAIDDATGKSPDRYSGDDRQATNAKVIKESSYYQDNLNNDKKVVNFFVAKDGSTKEDQLVDALAAAPVAANFGVTLNSDGKPVDKDGKVLTGSDNDKNKLVSPAPIVLATDSLSSDQSVSISKVLDKDNGENLVQVGKGIATSVINKLKDLLSM

SEQ ID NO: 4 - DNA, 2145 bp, Clostridium difficile
atgaataagaaaaacttagcaatggctatggcagcagttactgttgtgggttctgcagcgccaatatttgcagatagtactacgccaggttatactgtagtgaaaaatgattggaaaaaagcagtaaaacaattacaagatgggttgaaaaataaaactatatcaacaataaaggtgtcttttaatggaaactctgttggagaagttacaccagccagttctggagcaaaaaaagcagatagagatgctgcagctgaaaagttatataatttagtaaatacacaattagataaactaggtgatggagattacgttgactttgaagtaacttataatttagctactcaaataattacaaaagcagaagcagaggcagttcttacaaaattacaacaatataatgataaagtacttataaattctgcaacagatacagtaaaaggtatggtatctgatacacaagttgatagcaaaaatgttgcagctaacccacttaaagttagtgatatgtatacaataccatctgctattactggaagtgatgattctgggtatagtattgctaaaccaacagaaaagactacaagtttattgtatggtacggttggtgatgcaactgcaggtaaagcaataacagtagatacagcttcaaatgaagcttttgctggaaatggaaaggttattgactacaataaatcattcaaagcaactgtacaaggagatggaacagttaagacaagcggggttgtacttaaagatgcaagtgatatggctgcaacaggtactataaaagttagagttacaagtgcaaaagaagaatctattgatgtggattcaagttcatatattagtgctgaaaatttagctaaaaaatatgtatttaatcctaaagaggtttctgaagcttataatgcaatagttgcattacaaaatgatggaatagaatctgatttagtacaattagttaatggaaaatatcaagttattttctatccagaaggaaaaagattagaaactaaatctgcagatataatagctgatgcagatagtccagctaaaataactataaaagctaataaattaaaagatttaaaagattatgtagatgatttaaaaacatacaataatacttactcaaatgttgtaacagtagcaggagaagatagaatagaaactgctatagaattaagtagtaaatattataattctgatgataaaaatgcaataactgatgatgcagttaataatatagtattagttggatctacatctatagttgatggtcttgttgcatcaccattagcttcagaaaaaacagctccattattattaacttcaaaagataaattagattcatcagtaaaatctgagataaaaagagttatgaacttaaagagtgatactggtataaatacttctaaaaaagtttatttagctggtggagttaattctatatctaaagatgtagaaaatgaattgaaaaatatgggccttaaagttactagattatcaggagaagacagatacgaaacttctttagcaatagctgatgaaataggtcttgataatgataaagcatttgtagttggtggtactggattagcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgacagacaagcaactaatgctgaagttttaaaagaagatgattatttcaaagatggtgaagttgtgaattactttgttgcaaaagatggttctactaaagaagatcaattagtagatgcattagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatgtaa

SEQ ID NO: 5 - DNA, 2160 bp, Clostridium difficile
atgaataagaaaaatatagcaatagctatgtcaggtttaacagttttagcttcggctgctcctgtttttgctgcaactactggaacacaaggttatactgtagttaaaaacgactggaaaaaagcagtaaaacaattacaagatggactaaaagataatagtataggaaagataactgtatcttttaatgatggggttgtgggtgaagtagctcctaaaagtgctaataagaaagcggacagagatgctgcagctgagaagttatataatcttgttaacactcaattagataaattaggtgatggagattatgttgatttttctgtagattataatttagaaaacaaaataataactaatcaagcagatgcagaagcaattgttacaaagttaaattcacttaatgagaaaactcttattgatatagcaactaaagatacttttggaatggttagtaaaacacaagatagtgaaggtaaaaatgttgctgcaacaaaggcacttaaagttaaagatgttgctacatttggtttgaagtctggtggaagcgaagatactggatatgttgttgaaatgaaagcaggagctgtagaggataagtatggtaaagttggagatagtacggcaggtattgcaataaatcttcctagtactggacttgaatatgcaggtaaaggaacaacaattgattttaataaaactttaaaagttgatgtaacaggtggttcaacacctagtgctgtagctgtaagtggttttgtaactaaagatgatactgatttagcaaaatcaggtactataaatgtaagagttataaatgcaaaagaagaatcaattgatatagatgcaagctcatatacatcagctgaaaatttagctaaaagatatgtatttgatccagatgaaatttctgaagcatataaggcaatagtagcattacaaaatgatggtatagagtctaatttagttcagttagttaatggaaaatatcaagtgattttttatccagaaggtaaaagattagaaactaaatcagcaaatgatacaatagctagtcaagatacaccagctaaagtagttataaaagctaataaattaaaagatttaaaagattatgtagatgatttaaaaacatataataatacttattcaaatgttgtaacagtagcaggagaagatagaatagaaactgctatagaattaagtagtaaatattataattctgatgataaaaatgcaataactgataaagcagttaatgatatagtattagttggatctacatctatagttgatggtcttgttgcatcaccattagcttcagaaaaaacagctccattattattaacttcaaaagataaattagattcatcagtaaaatctgaaataaagagagttatgaacttaaagagtgacactggtataaatacttctaaaaaagtttatttagctggtggagttaattctatatctaaagatgtagaaaatgaattgaaaaacatgggtcttaaagttactagattatcaggagaagacagatacgaaacttctttagcaatagctgatgaaataggtcttgataatgataaagcatttgtagttggtggtactggattagcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgatagacaagcaactaatgctgaagttttaaaagaagatgattatttcacagatggtgaagttgtgaattactttgttgcaaaagatggttctactaaagaagatcaattagtagatgccttagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatgtaa

SEQ ID NO: 6 - DNA, 2271 bp, Clostridium difficile
atgaataagaaaaatatagcaatagctatgtcaggtttaacagttttagcttcggctgcacctgtatttgcagatgatacaaaagttgaaactggtgatcaaggatatacagtggtacaaagcaagtataagaaagctgttgaacaattacaaaaaggaatattagatggaagtataacagaaattaaagttttctttgagggaactttagcatctactataaaagtaggttctgagcttaatgcagcagatgcaagtaaattattgtttacacaagtagataataaactagataatttaggtgatggagattatgtagatttcttaataacttctccaggtcaaggggataaaataactacaagtaaacttgttgcattgaaagatttaacaggtgcttcagcagatgctataattgctggaacatcttcagcagatggtgttgttacaaatactggagctgctagtggttctactgagacaaattcagcaggaacaaaacttgcaatgtcagctatttttgacacagcatatacagattcatctgaaactgcggttaagattactataaaagcagatatgaatgatactaaatttggtaaagcaggtgagacaacttattcaactgggcttacatttgaagatgggtctacagaaaaaattgttaaattaggggacagtgatattatagatataactaaagctcttaaacttactgttgttcctggaagtaaagcaactgttaagtttgctgaaaaaacaccaagtgccagtgttcaaccagtaataacaaagcttagaataataaatgctaaagaagaaacaatagatattgacgctagttctagtaaaacagcacaagatttagctaaaaaatatgtatttaataaaactgatttaaatactctttataaagtattaaatggagatgaagcagatactaatggattaatagaagaagttagtggaaaatatcaagtagttctttatccagaaggaaaaagagttacaactaagagtgctgcaaaggcttcaattgctgatgaaaattcaccagttaaattaactcttaagtcagataagaagaaagacttaaaagattatgtggatgatttaagaacatataataatggatattcaaatgctatagaagtagcaggagaagatagaatagaaactgcaatagcattaagtcaaaaatattataactctgatgatgaaaatgctatatttagagattcagttgataatgtagtattggttggaggaaatgcaatagttgatggacttgtagcttctcctttagcttctgaaaagaaagctcctttattattaacttcaaaagataaattagattcaagcgtaaaagctgaaataaagagagttatgaatataaagagtacaacaggtataaatacttcaaagaaagtttatttagctggtggagttaattctatatctaaagaagtagaaaatgaattaaaagatatgggacttaaagttacaagattagcaggagatgatagatatgaaacttctctaaaaatagctgatgaagtaggtcttgataatgataaagcatttgtagttggaggaacaggattagcagatgccatgagtatagctccagttgcatctcaattaagaaatgctaatggtaaaatggatttagctgatggtgatgctacaccaatagtagttgtagatggaaaagctaaaactataaatgatgatgtaaaagatttcttagatgattcacaagttgatataataggtggagaaaacagtgtatctaaagatgttgaaaatgcaatagatgatgctacaggtaaatctccagatagatatagtggagatgatagacaagcaactaatgcaaaagttataaaagaatcttcttattatcaagataacttaaataatgataaaaaagtagttaatttctttgtagctaaagatggttctactaaagaagatcaattagttgatgctttagcagcagctccagttgcagcaaactttggtgtaactcttaattctgatggtaagccagtagataaagatggtaaagtattaactggttctgataatgataaaaataaattagtatctccagcacctatagtattagctactgattctttatcttcagatcaaagtgtatctataagtaaagttcttgataaagataatggagaaaacttagttcaagttggtaaaggtatagctacttcagttataaacaaattaaaagatttattaagtatgtaa

SEQ ID NO: 7 - protein, 23 aa, Clostridium difficile
  MISC_FEATURE (21)..(21): UNCERTAIN AMINO ACID
AAKASIADENSPVKLTLKSDXKX

SEQ ID NO: 8 - protein, 30 aa, Clostridium difficile
  MISC_FEATURE (26)..(26): POSSIBLY CYSTINE BUT UNCERTAIN
ADIIADADSPAKITIKANKLKDLKDXVDDL

SEQ ID NO: 9 - protein, 30 aa, Clostridium difficile
DDTKVETGDQGYTVVQSKKYKAAVEQLQKI

SEQ ID NO: 10 - protein, 14 aa, Clostridium difficile
DSTTPGVVTVVKND

SEQ ID NO: 11 - protein, 9 aa, Clostridium difficile
YYNSDDENA

SEQ ID NO: 12 - protein, 10 aa, Clostridium difficile
  MISC_FEATURE (6)..(6): EITHER LEUCINE OR ISOLEUCINE
VGGTGXADAM

SEQ ID NO: 13 - protein, 6 aa, Clostridium difficile
  MISC_FEATURE (5)..(5): EITHER LEUCINE OR ISOLEUCINE
YQVVXY

SEQ ID NO: 14 - protein, 9 aa, Clostridium difficile
  MISC_FEATURE (5)..(5): EITHER LEUCINE OR ISOLEUCINE
VGSEXNAAD

SEQ ID NO: 15 - protein, 7 aa, Clostridium difficile
  MISC_FEATURE (4)..(4): EITHER LEUCINE OR ISOLEUCINE
VDAXAAA

SEQ ID NO: 16 - protein, 8 aa, Clostridium difficile
  MISC_FEATURE (3)..(3): EITHER LEUCINE OR ISOLEUCINE
VYXAGGVN

SEQ ID NO: 17 - protein, 6 aa, Clostridium difficile
  MISC_FEATURE (4)..(4): EITHER LEUCINE OR ISOLEUCINE
YQVXFY

SEQ ID NO: 18 - protein, 15 aa, Clostridium difficile
TVDTASNEAFAGDGK

(19) United States
(12) Patent Application Publication
(10) Pub. No.: US 2004/0039165 A1
(43) Pub. Date: Feb. 26, 2004

(54) Clostridium difficile polypeptides and uses thereof

(76) Inventors: Neil Fraser Fairweather, Kent (GB); Emanuela Calabi, Lecce (IT)

Correspondence Address: Scott A McCollister, Fay Sharpe Fagan Minnich & McKee, 7th Floor, 1100 Superior Avenue, Cleveland, OH, 44114-2518, US

(21) Appl. No.: 10/239,610

(22) PCT Filed: Mar. 23, 2001
(86) PCT No.: PCT/GB01/01305

(30) Foreign Application Priority Data
Mar. 24, 2000 (GB) 0007263.7

(51) Int. Cl. C07K 1/00; C07K 14/00; C07K 17/00

(52) U.S. Cl. 530/350

(57) ABSTRACT

The present invention relates to a polypeptide comprising the amino acid sequence shown in SEQ ID No. 1, SEQ ID No. 2 or SEQ ID No. 3, or a homologue, variant or derivative thereof; a peptide comprising a portion of such a polypeptide; and a polynucleotide capable of encoding such a polypeptide.

### FIELD OF THE INVENTION

The present invention relates to new polypeptides and new polynucleotides.

In particular, the invention relates to new polypeptides derivable from C. difficile, and new polynucleotides capable of encoding the new polynucleotides.

The present invention also relates to peptides derivable from the polynucleotides, and various uses of the polypeptides, polynucleotides and peptides in prophylactic and therapeutic applications.

### BACKGROUND TO THE INVENTION

Clostridium difficile

Clostridium difficile, a Gram-positive anaerobic bacterium, is a major cause of antibiotic associated diarrhoea and pseudomembranous colitis in humans. These conditions commence with colonisation of the large intestine by C. difficile, and the subsequent production of toxins is thought to mediate much of the subsequent tissue and cellular damage. Infections are associated with intensive antibiotic treatment during hospitalisation, which may alter the resident microflora allowing proliferation of C. difficile. In severe outbreaks, infections can result in ward closure, causing extreme inconvenience and expense to the health service (reviewed in Bartlett, J. G. (1979) Rev Infect Dis. 1(3):530-9).

There are currently no effective vaccines against C. difficile. The majority of research to date on C. difficile has concentrated on the two large toxins, toxin A (308 kDa) and toxin B (270 kDa), which are clearly virulence factors of this pathogen (for a review, see von Eichel-Streiber, C., et al (1996) Trends Microbiol 4(10), 375-82). Experimental vaccines based on chemically or genetically modified toxins have been studied in animal models of infection and show limited effectiveness. There is therefore a need for alternative and improved vaccines against C. difficile.

S-layers

S-layers are a feature present in many bacterial species. The functions of S-layers are varied, but their contribution to virulence has been investigated, for example in Camplyobacter fetus. In C. difficile, the S-layer proteins are the predominant cell wall protein and, like those from other species, form an ordered structure on the outer surface of the bacterium (Takeoka et al (1991) J. Gen. Microbiol. 137 (Pt 2), 261-267). The S-layer of C. difficile is composed of two distinct proteins which vary in size between strains, although in most strains one protein is between 45-50 kDa and another is 30-40 kDa in size. For example, in the study by Takeoka (Takeoka et al (1991)—as above), the two proteins which represented the S-layer are found to be 32 kDa and 35 kDa. In a separate study using C. difficile strain C253, an immunodominant antigen of 36 kDa is found which probably represents a component of the S-layer (Cerquetti et al (1992) Microb. Pathog. 13(4) 271-279). No amino acid sequence data is available on C. difficile S-layer genes, nor have the genes been identified.

### SUMMARY OF ASPECTS OF THE PRESENT INVENTION

The present inventors have found that the two cell wall proteins found in C. difficile strains, and which are believed to represent the S-layer proteins, are synthesised as one polypeptide which is then subjected to cleavage to produce the two protein species characteristic of C. difficile cell walls. The present inventors have elucidated and compared the gene and protein sequences for this polypeptide from three strains of C. difficile.

In a first aspect, the present invention provides a polypeptide. The polypeptide may be derivable from C. difficile, for example from the cell wall, or more particularly the S-layer of C. difficile.

In particular, the polypeptide may comprise the amino acid sequence shown in SEQ ID No. 1, SEQ ID No. 2 or SEQ ID No. 3, or a homologue, variant or derivative thereof.

Here, SEQ ID No. 1, SEQ ID No. 2 or SEQ ID No. 3 are either the sequences respectively presented as SEQ ID No. 1, SEQ ID No. 2 or SEQ ID No. 3 in the following Sequences Listings Section or the sequences presented in FIG. 1. Here, FIG. 1 shows a sequence comparison (alignment) between the cell wall proteins of three strains of C. difficile, namely “17” (714 amino acids), “630” (719 amino acids) and “1” (756 amino acids). In FIG. 1, SEQ ID No.1 is the top sequence (“17”); SEQ ID NO. 2 is the middle sequence (“630”); and SEQ ID No. 3 is the bottom sequence (“1”).

Preferably, SEQ ID No. 1, SEQ ID No. 2 or SEQ ID No. 3 are the sequences respectively presented as SEQ ID No. 1, SEQ ID No. 2 or SEQ ID No. 3 in the following Sequences Listings Section.

In a second aspect, the present invention provides a polynucleotide. The polynucleotide may be derivable from C. difficile. The polynucleotide may encode a cell wall protein (particularly an S-layer cell wall protein) or part thereof. The polynucleotide may be capable of encoding a polypeptide of the first aspect of the invention.

In one embodiment, the polynucleotide comprises the nucleic acid sequence shown in SEQ ID No. 4, SEQ ID No. 5 or SEQ ID No. 6, or a homologue, variant or derivative thereof.

Here, SEQ ID No. 4, SEQ ID No. 5 or SEQ ID No. 6 are either the sequences respectively presented as SEQ ID No. 4, SEQ ID No. 5 or SEQ ID No. 6 in the following Sequences Listings Section or the sequences presented in FIG. 2. Here, FIG. 2 shows an alignment between the DNA sequences of the S-layer genes of C. difficile strains “17” (2145 bp) “630” (2160 bp) and “1” (2271 bp). In FIG. 2, SEQ ID No.4 is the top sequence (“17”); SEQ ID NO. 5 is the middle sequence (“630”); and SEQ ID No. 6 is the bottom sequence (“1”).

Preferably, SEQ ID No. 4, SEQ ID No. 5 or SEQ ID No. 6 are the sequences respectively presented as SEQ ID No. 4, SEQ ID No. 5 or SEQ ID No. 6 in the following Sequences Listings Section.

In a third aspect, the present invention provides a peptide. The peptide may comprise a portion of a polypeptide of the first aspect of the invention.

In a fourth aspect, the present invention provides a nucleotide. The nucleotide may encode a peptide according to the third aspect of the invention. The nucleotide may be derivable from C. difficile.

In a fifth aspect, the present invention provides a vector comprising such a polynucleotide or nucleotide. The polynucleotide or nucleotide may be linked to a regulatory sequence. Preferably the regulatory sequence allows expression of the polynucleotide or nucleotide in a host cell.

In a sixth aspect, the present invention provides a host cell comprising such a vector.

In a seventh aspect, the present invention provides a method for screening for a compound. The method may comprise the step of using a polypeptide or peptide according to an earlier aspect of the invention. The compound may be capable of interacting specifically with a C. difficile S-layer protein.

In an eighth aspect, the present invention provides a compound. The compound may be capable of binding specifically to a polypeptide and/or a peptide according to an earlier aspect of the invention.

In a ninth aspect, the present invention provides the use of a polypeptide, polynucleotide, peptide, or nucleotide according to an earlier aspect of the invention in a method for producing antibodies.

In a tenth aspect, the present invention provides an antibody. The antibody may be capable of binding specifically to a polypeptide and/or peptide according to an earlier aspect of the invention.

In an eleventh aspect, the present invention provides a pharmaceutical composition. The pharmaceutical composition may comprise any one of more of a polypeptide, polynucleotide, peptide, vector or antibody according to earlier aspects of the invention.

In a twelfth aspect, the present invention provides a composition that is capable of inducing an immune response—such as a vaccine composition. Here, the composition may be termed an immune modulating composition. Preferably, said immune modulating composition is a vaccine. The immune modulating composition may comprise any one of more of a polypeptide, polynucleotide, peptide, vector or antibody according to earlier aspects of the invention.

In a thirteenth aspect, the present invention provides a method for treating and/or preventing a disease in a subject. The method may comprise the step of administering any one of more of a polypeptide, polynucleotide, peptide, vector, antibody, pharmaceutical composition or immune modulating composition according to earlier aspects of the invention to the subject. In a preferred embodiment the method of the thirteenth aspect of the invention is used to treat and/or prevent a disease which is associated with Clostridium difficile infection.

### DETAILED ASPECTS OF THE INVENTION

Although in general the techniques mentioned herein are well known in the art, reference may be made in particular to Sambrook et al., Molecular Cloning, A Laboratory Manual (1989) and Ausubel et al., Current Protocols in Molecular Biology (1995), John Wiley & Sons, Inc.

### POLYPEPTIDES AND PEPTIDES

The amino acid sequences of the S-layer protein from three strains of C. difficile have been determined. The polypeptide of the first aspect of the invention may comprise one these amino acid sequences (as shown in SEQ ID No. 1, SEQ ID No. 2 or SEQ ID No. 3), or a homologue, variant or derivative thereof. In a preferred embodiment, the polypeptide of the first aspect of the invention comprises the amino acid sequences shown in SEQ ID No. 1 or SEQ ID No. 3, or a homologue, variant or derivative thereof.

In addition to cell wall proteins from the three strains of C. difficile presented herein (SEQ ID No. 1 or SEQ ID No. 2 or SEQ ID No. 3), the first aspect of the invention also includes homologous sequences obtained from other strains of C. difficile, or from other related bacteria. Moreover, the first aspect of the present invention includes a homologous sequence isolated from any source, as well as synthetic amino acid sequences.

In the context of the present invention, a homologous sequence is taken to include an amino acid sequence which may be at least 75, 85 or 90% identical, preferably at least 95 or 98% identical at the amino acid level to one of the three sequences shown as SEQ ID No. 1 or SEQ ID No. 2 or SEQ ID No. 3. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the polypeptide of the first aspect of the invention includes any substitution of, variation of, modification of, replacement of, deletion of or addition of one (or more) amino acids from the amino acid sequences of SEQ ID No. 1, SEQ ID No. 2, or Seq ID No. 3, or a homologue thereof.

The polypeptide of the present invention may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides of the invention may further comprise heterologous amino acid sequences, typically at the N-terminus or C-terminus, preferably the N-terminus. Heterologous sequences may include sequences that affect intra or extracellular protein targeting (such as leader sequences). Heterologous sequences may also include sequences that increase the immunogenicity of the polypeptide of the invention and/or which facilitate identification, extraction and/or purification of the polypeptides. Another heterologous sequence that is particularly preferred is a polyamino acid sequence such as polyhistidine which is preferably N-terminal. A polyhistidine sequence of at least 10 amino acids, preferably at least 17 amino acids but fewer than 50 amino acids is especially preferred.

Other heterologous amino acid sequences includes immunogenic sequences from other pathogenic organisms such as bacteria or viruses. Examples include pathogenic E. coli Neiserria sp., B. pertussis, C. difficile, Salmonella sp., Campylobacter sp., P. falciparum, hepatitis B virus, hepatitis C virus and human papilloma virus.

Polypeptides of the invention are typically made by recombinant means, using known techniques. However they may also be made by synthetic means using techniques well known to skilled persons such as solid phase synthesis. Polypeptides of the invention may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site.

Preferably the fusion protein will not hinder the function of the protein of interest sequence.

Polypeptides of the invention may be in a substantially isolated form. It will be understood that the protein may be mixed with carriers or diluents which will not interfere with the intended purpose of the protein and still be regarded as substantially isolated. A polypeptide of the invention may also be in a substantially purified form, in which case it will generally comprise the protein in a preparation in which more than 90%, e.g. 95%, 98% or 99% of the protein in the preparation is a polypeptide of the invention.

The present invention also relates to peptides comprising a portion of the polypeptide of the first aspect of the invention.

The peptides of the present invention may be between 2 and 200 amino acids, preferably between 4 and 40 amino acids in length.

The peptide may be derived from a polypeptide of the first aspect of the invention, for example by digestion with a suitable enzyme, such as trypsin. Alternatively the peptide may be made by recombinant means, or synthesised synthetically,

The term “peptide” includes the various synthetic peptide variations known in the art, such as a retroinverso D peptides.

The peptide may be an antigenic determinant and/or a T-cell epitope. The peptide may be immunogenic in vivo. Preferably the peptide is capable of inducing neutralising antibodies in vivo.

The present inventors have shown that there is considerable amino acid variation in amino acid sequence between cell wall proteins from different strains of C. difficile. By aligning these sequences, it is possible to determine which regions of the amino acid sequence are conserved between different strains (“homologous regions”), and which regions vary between the different strains (“heterologous regions”).

The peptide of the present of the invention may comprise a sequence which corresponds to at least part of a homologous region. A homologous region shows a high degree of homology between at least two strains of C. difficile. For example, the homologous region may show at least 80%, preferably at least 90%, more preferably at least 95% identity at the amino acid level using the tests described above. Peptides which comprise a sequence which corresponds to a homologous region may be used in therapeutic strategies aimed at more than one strain of C. difficile. For example, a vaccine comprising such a peptide could be used to induce a protective immune response to each or every strain of C. difficile which shows a high degree of homology to the peptide sequence in that particular region. If the homologous region is conserved (i.e. shows a high degree of homology) between all strains of C. difficile, the peptide may be used to design vaccines against all strains.

Alternatively, the peptide of the second aspect of the invention may comprise a sequence which corresponds to at least part of a heterologous region. A heterologous region shows a low degree of homology between at least two strains of C. difficile. For example, the heterologous region may show less than 60%, preferably less than 50%, more preferably less than 40% identity at the amino acid level using the tests described above. Peptides which comprise a sequence which corresponds to a heterologous region may be used in therapeutic strategies aimed a particular strain of C. difficile. For example, a vaccine comprising such a peptide could be used to induce a protective immune response to a particular strain of C. difficile which (unlike other strains) shows a high degree of homology to the peptide sequence in that particular region.

Nucleotides and Polynucleotides

The present invention also provides nucleic acid sequences capable of encoding the polypeptides and peptides of the present invention. It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. Preferably, the term nucleotide sequence is prepared by use of recombinant DNA techniques (e.g. recombinant DNA).

Preferably, the term “nucleotide sequence” means DNA.

The terms “variant”, “homologue” or “derivative” in relation to the nucleotide sequence of the second aspect of the present invention include any substitution of, variation of, modification of, replacement of, deletion of or addition of one (or more) nucleic acid from or to the sequence providing the resultant nucleotide sequence codes for an amino acid sequence according to the first aspect of the invention.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above.

As used herein a “deletion” is defined as a change in either nucleotide or amino acid sequence in which one or more nucleotides or amino acid residues, respectively, are absent.

As used herein an “insertion” or “addition” is that change in a nucleotide or amino acid sequence which has resulted in the addition of one or more nucleotides or amino acid residues, respectively, as compared to the naturally occurring substance.

As used herein “substitution” results from the replacement of one or more nucleotides or amino acids by different nucleotides or amino acids, respectively.

The term “hybridization” as used herein shall include “the process by which a strand of nucleic acid joins with a complementary strand through base pairing” as well as the process of amplification as carried out in polymerase chain reaction (PCR) technologies.

Nucleotide sequences of the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to one of the three nucleotide sequences shown as SEQ ID No. 4 or SEQ ID No. 5 or SEQ ID No. 6, preferably at least 80 or 90% and more preferably at least 95% homologous to one of the sequences.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Expression Vectors

Polynucleotides and nucleotides of the invention can be incorporated into a recombinant replicable vector. The vector may be used to replicate the nucleic acid in a compatible host cell. Thus, in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a regulatory sequence that is capable of providing for the expression of the coding sequence by the host cell, i.e. the vector is an expression vector. The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

Such vectors may be transformed or transfected into a suitable host cell to provide for expression of a polypeptide of the invention. Suitable host cells include prokaryotes such as eubacteria, for example E. coli and B. subtilis and eukaryotes such as yeast, insect or mammalian cells.

Vectors/polynucleotides of the invention may be introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

The transformed host cell may be cultured under conditions to provide for expression by the vector of a coding sequence encoding the polypeptide or peptide, and optionally recovering the expressed polypeptide or peptide.

The vectors may be, for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in a method of gene therapy.

Promoters/enhancers and other expression regulation signals may be selected to be compatible with the host cell for which the expression vector is designed For example, prokaryotic promoters may be used, in particular those suitable for use in E. coli strains (such as E. coli HB 101). In a particularly preferred embodiment of the invention, an htrA or nirB promoter may be used. When expression of the polypeptides of the invention is carried out in mammalian cells, either in vitro or in vivo, mammalian promoters may be used. Tissue-specific promoters may also be used. Viral promoters may also be used, for example the Moloney murine leukaemia virus long terminal repeat (MMLV LTR), the promoter rous sarcoma virus (RSV) LTR promoter, the SV40 promoter, the human cytomegalovirus (CMV) IE promoter, herpes simplex virus promoters or adenovirus promoters. All these promoters are readily available in the art.

The vector, polynucleotide or nucleotide of the present invention may be delivered by a viral or a non-viral method. Viral delivery systems include but are not limited to adenovirus vector, an adeno-associated viral (AAV) vector, a herpes viral vector, retroviral vector, lentiviral vector, baculoviral vector.

Non viral delivery systems include: DNA transfection methods of, for example, plasmids, chromosomes or artificial chromosomes. Here transfection includes a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) Nature Biotechnology 1996 14; 556), and combinations thereof.

Non-viral delivery systems also include peptide delivery which uses domains or sequences from proteins capable of translocation through the plasma and/or nuclear membrane

Alternatively amino acid sequences or nucleic acid sequences may be directly introduced to the cell by microinjection, or delivery using vesicles such as liposomes which are capable of fusing with the cell membrane. Viral fusogenic peptides may also be used to promote membrane fusion and delivery to the cytoplasm of the cell.

Screening Systems

The present invention also provides a method for screening for a compound which is capable of interacting specifically with a C. difficile S-layer protein. For example, the method may be used to screen a plurality of compounds in the form of a library.

Where the candidate compounds are proteins, in particular antibodies or peptides, libraries of candidate compounds can be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a polypeptide or peptide of the invention are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Anther method of screening a library of compounds utilises eukaryotic or prokaryotic host cells which are stably transformed with recombinant DNA molecules expressing the library of compounds. Such cells, either in viable or fixed form, can be used for standard binding-partner assays. See also Parce et al. (1989) Science 246:243-247; and Owicki et al. (1990) Proc. Nat'l Acad. Sci. USA 87;4007-4011, which describe sensitive methods to detect cellular responses. Competitive assays are particularly useful, where the cells expressing the library of compounds are contacted incubated with a labelled antibody known to bind to a polypeptide of the present invention, such as ¹²⁵I-antibody, and a test sample such as a candidate compound whose binding affinity to the binding composition is being measured. The bound and free labelled binding partners for the polypeptide are then separated to assess the degree of binding.

The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a polypeptide of the invention, and is described in detail in International Patent application no. WO 84/03564 (Cornrnonwealth Serum Labs.), published on Sep. 13, 1984. First, large numbers of different small peptide test compounds are synthesized on a solid substrate, e.g., plastic pins or some other appropriate surface; see Fodor et al. (1991). Then all the pins are reacted with solubilized polypeptide of the invention and ished. The next step involves detecting bound polypeptide. Compounds which interact specifically with the polypeptide will thus be identified.

Rational design of candidate compounds likely to be able to interact with C. difficile S-layer protein may be based upon structural studies of the molecular shapes of a polypeptide of the first apsect of the invention. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

The present invention also provides a compound capable of binding specifically to a polypeptide and/or peptide of the present invention.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule.

Preferably the compound is an antibody.

Antibodies

For the purposes of this invention, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies, for example as described in substance-A-239400. Neutralizing antibodies, i.e., those which inhibit biological activity of the substance amino acid sequences, are especially preferred for diagnostics and therapeutics.

Antibodies may be produced by standard techniques, such as by immunisation or by using a phage display library.

A polypeptide or peptide of the present invention may be used to develop an antibody by known techniques. Such an antibody may be capable of binding specifically to the S-layer protein of C. difficile.

If polyclonal antibodies are desired, a selected mammal (e.g., mouse, rabbit, goat, horse, etc.) may immunised with an immunogenic composition comprising a polypeptide or peptide of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance amino acid sequence is administered to immunologically compromised individuals for the purpose of stimulating systemic defence.

Serum from the immunised animal is collected and treated according to known procedures. If serum containing polyclonal antibodies to an epitope obtainable from a polypeptide of the present invention contains antibodies to other antigens, the polyclonal antibodies can be purified by immunoaffinity chromatography. Techniques for producing and processing polyclonal antisera are known in the art. In order that such antibodies may be made, the invention also provides amino acid sequences of the invention or fragments thereof haptenised to another amino acid sequence for use as immunogens in animals or humans.

Monoclonal antibodies directed against epitopes obtainable from a polypeptide or peptide of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from a polypeptide or peptide of the present invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the polypeptide or peptide may also be generated. For example, such fragments include, but are not limited to, the F(ab)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse WD et al (1989) Science 256:1275-1281).

Pharmaceutical Compositions

The present invention also provides a pharmaceutical composition comprising administering a therapeutically effective amount of the polypeptide, polynucleotide, peptide, vector or antibody of the present invention and optionally a pharmaceutically acceptable carrier, diluent or excipients (including combinations thereof). The pharmaceutical composition of the present invention may also contain or may be used in conjunction with one or more additional pharmaceutically active compounds and/or adjuvants.

The pharmaceutical compositions may be for human or animal usage in human and veterinary medicine and will typically comprise any one or more of a pharmaceutically acceptable diluent, carrier, or excipient Acceptable carriers or diluents for therapeutic use are well known in the pharmaceutical art, and are described, for example, in Remington's Pharmaceutical Sciences, Mack Publishing Co. (A. R. Gennaro edit 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Vaccines

Preferably the immune modulating composition is a vaccine.

Vaccines may be prepared from one or more polypeptides or peptides of the present invention.

The preparation of vaccines which contain an immunogenic polypeptide(s) or peptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Proplonobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The polypeptides of the invention may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The pharmaceutical and immune modulating compositions of the present invention may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

The term “co-administered” means that the site and time of administration of each of for example, the polypeptide of the present invention and an additional entity such as adjuvant are such that the necessary modulation of the immune system is achieved. Thus, whilst the polypeptide and the adjuvant may be administered at the same moment in time and at the same site, there may be advantages in administering the polypeptide at a different time and to a different site from the adjuvant. The polypeptide and adjuvant may even be delivered in the same delivery vehicle—and the polypeptide and the antigen may be coupled and/or uncoupled and/or genetically coupled and/or uncoupled.

The polypeptide, polynucleotide, peptide, nucleotide, antibody of the invention and optionally an adjuvant may be administered separately or co-administered to the host subject as a single dose or in multiple doses.

The immune modulating composition and pharmaceutical composition of the present invention may be administered by a number of different routes such as injection (which includes parenteral, subcutaneous and intramuscular injection) intranasal, mucosal, oral, intra-vaginal, urethral or ocular administration.

The immune modulating composition and pharmaceutical composition of the present invention may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, may be 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the immune modulating composition is lyophilised, the lyophilised material may be reconstituted prior to administration, e.g. as a suspension. Reconstitution is preferably effected in buffer

Diseases

The present invention also provides a method for treating and/or preventing a disease which comprises the step of administering any one of more of a polypeptide, polynucleotide, peptide, vector, antibody, pharmaceutical composition or immune modulating composition according to earlier aspects of the invention to the subject.

The method is particularly suited to treating diseases associated with C. difficile. Preferably the method protects against colonisation of C. difficile. In this embodiment, the method may prevent or reverse the build up of toxins which cause the cell and tissue damage characteristic of C. difficile-associated diseases.

Clostridium difficile is the major causative agent of pseudomembranous colitis (PMC) in humans. PMC is characterized by diarrhoea, a severe inflammation of the colonic mucosa, and formation of pseudomembranes that are composed of fibrin, mucus, necrotic epithelial cells, and leukocytes. The pseudomembrane can form a sheath over the entire colonic mucosa. In addition to causing PMC, C. difficile is believed to play a role in other less severe gastrointestinal illnesses; the organism is estimated to cause approximately 25% of reported cases of antibiotic-associated diarrhoea (Brettle and Wallace (1984) J. Infect. 8: 123-128; Gilligan et al. (1981) J. Clin. Microbiol. 14: 26-31). C. difficile caused diseases are not limited to gastrointestinal illnesses, as the organism can cause abscesses, wound infections, osteomyelitis, urogenital tract infections, septicemia, peritonitis, and pleuritis (Lyerly et al. (1988) Clin. Microbiol. Rev. 1:1-18; Hafiz et al. (1975) Lancet 1: 420-421; Levett (1986) J. Infect. 12: 253-263; Saginur et al. (1983) J. Infect. Dis. 147: 1105). Antibiotics can predispose a host animal to PMC and other C. difficile-related illnesses, as the disturbance of the normal bacterial flora by the antibiotic disrupts the major barrier against colonization by pathogens, rendering the host animal susceptible to colonization by pathogens such as C. difficile. Hospitals and chronic care facilities are significant sources of C. difficile infection, with one study finding that 21% of patients acquired C. difficile infection during hospitalization (McFarland et al. (1989) N. Engl. J. Med. 320: 204).

Various methods for detecting C. difficile infection are known. One previously known method for detecting C. difficile infection is culture on agar media. A commercially available assay for C. difficile involves latex agglutination of an antigen that is eventually identified as C. difficile glutamate dehydrogenase. Lyerly et al. (1991) J. Clin. Microbiol. 29: 2639; Lyerly et al. (1986) J. Clin. Microbiol. 23: 622. U.S. Pat. No. 5,965,375 describes various methods, compositions, and kits for detecting the presence of toxigenic strains of C. difficile in a biological sample.

C. difficile strains can be typed on the basis of the profiles of bands obtained after PCR ribotyping (Stubbs, S. L. et al. (1999). J Clin Microbiol. 37(2):461-3), in addition to other known methods (Heard, S. R., et al (1986) J Infect Dis 153(1), 159-62; Delmee, M. et al (1985) J Clin Microbiol. 21(3):323-7).

### FIGURES

The present invention will now be described only by way of examples, in which reference is made to the following Figures:

FIG. 1, which presents a sequence alignment;

FIG. 2, which presents a sequence alignment;

FIG. 3, which presents a diagrammatic image;

FIG. 4, which presents a diagrammatic image;

FIG. 5, which presents a diagrammatic image;

FIG. 6, which presents a photographic image;

FIG. 7, which presents a photographic image;

FIG. 8, which presents a photographic image; and

FIG. 9, which presents photographic images.

### In more detail:

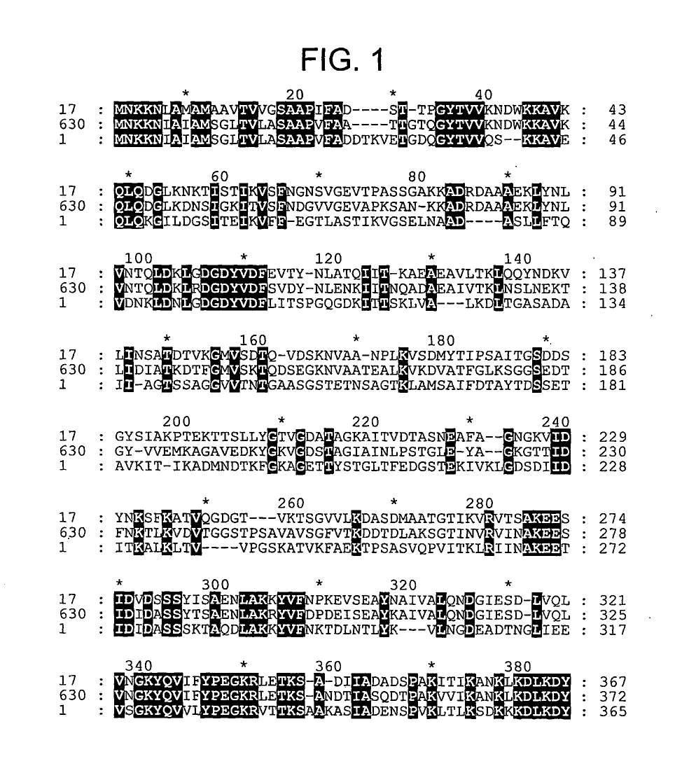

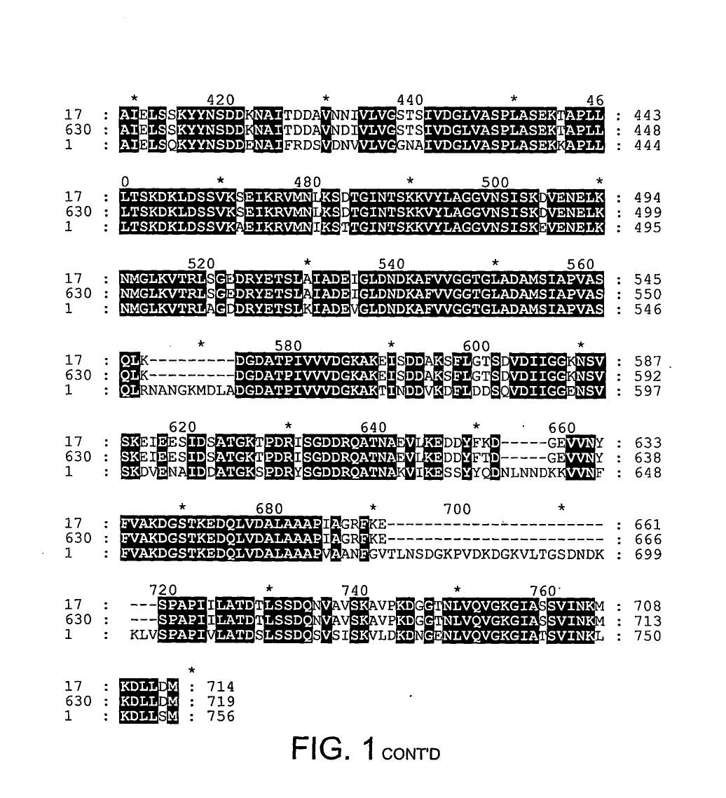

FIG. 1 shows an alignment between the amino acid sequences of the S-layer proteins of C. difficile strains 1, 17 and 630. Amino acid residues of the proteins in boxes are homologous in all three of the strains examined. Those not in boxes show no homology, or only homology between two out of three of the strains examined.

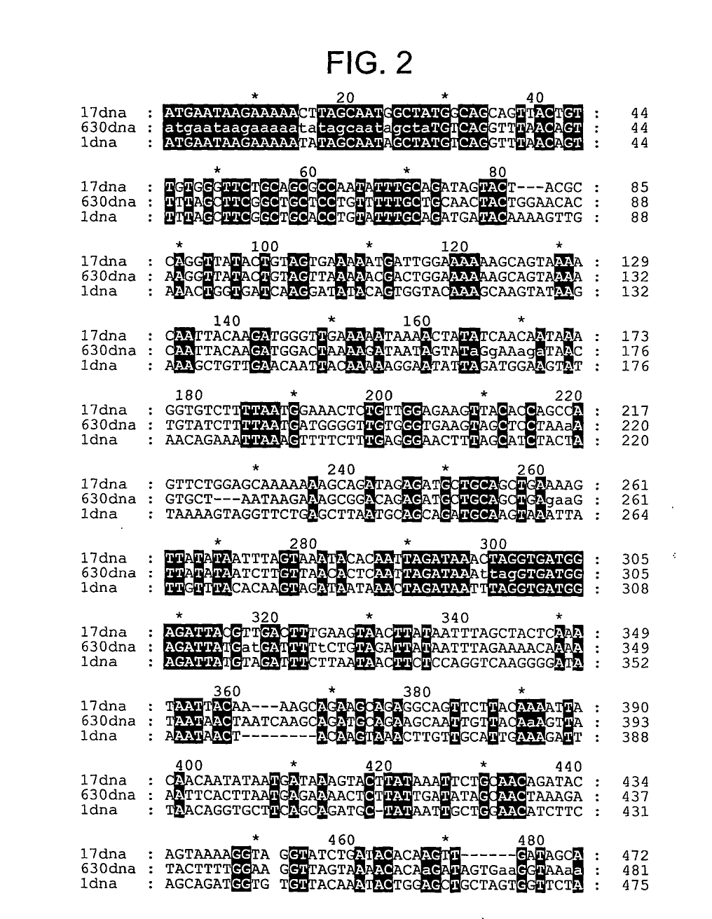

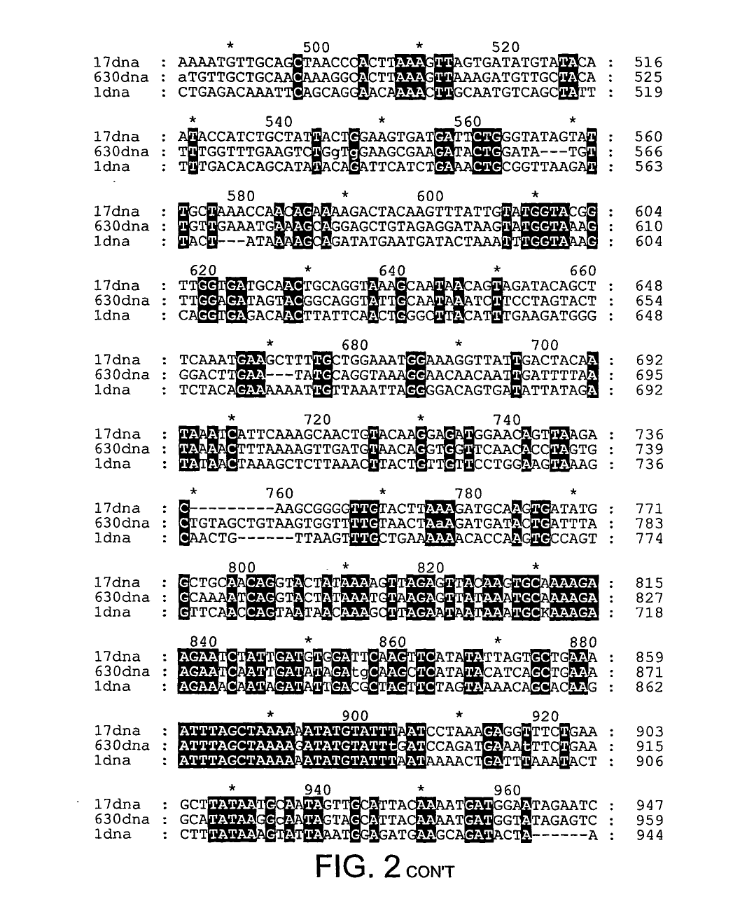

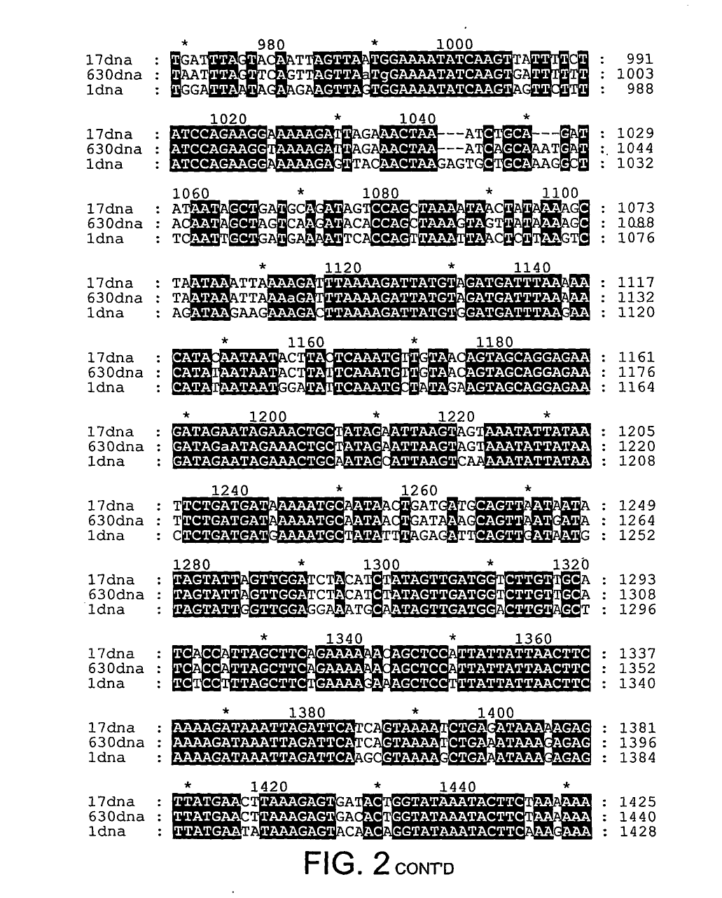

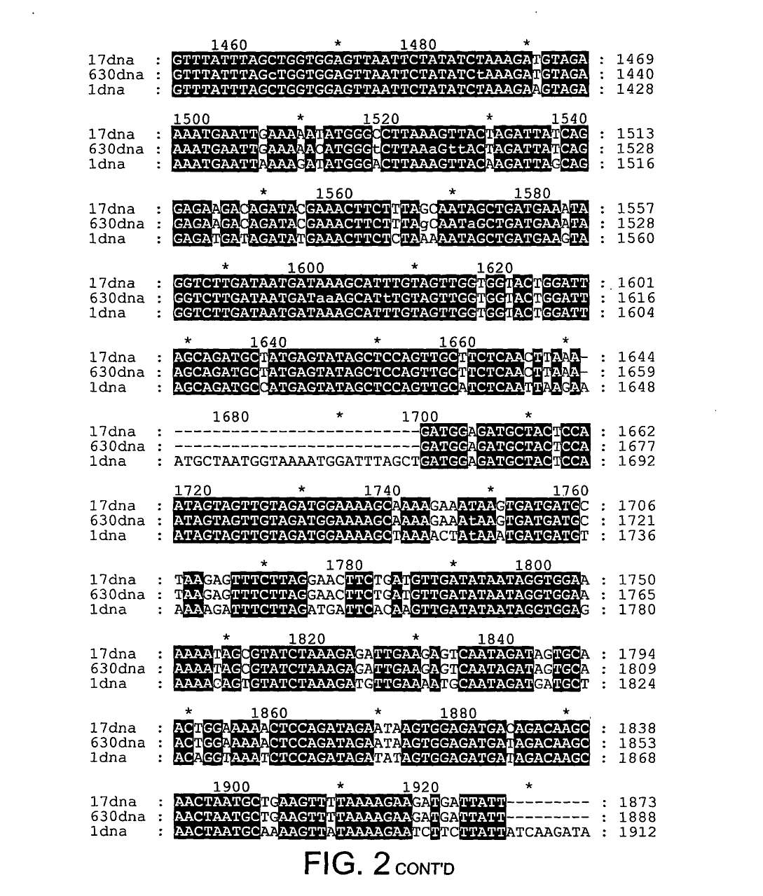

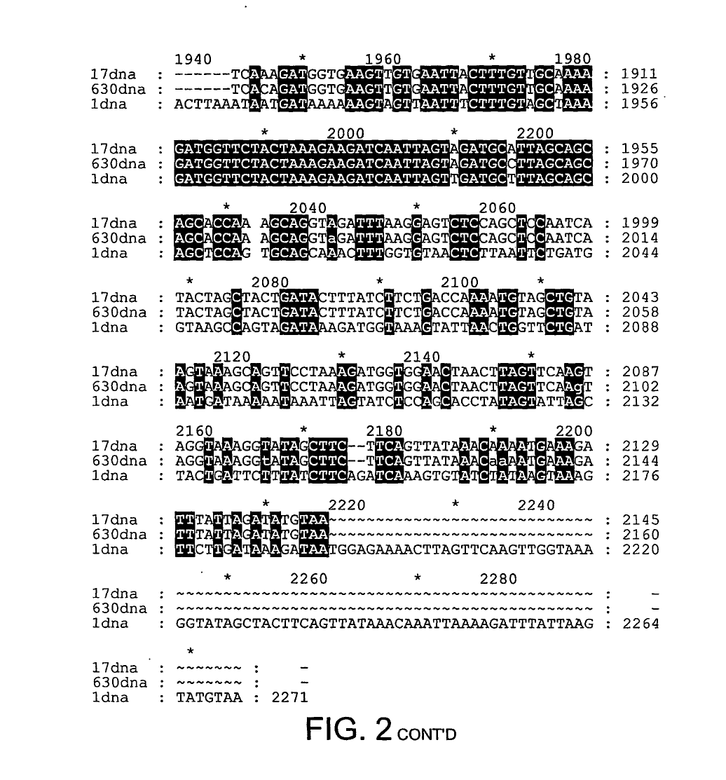

FIG. 2 shows an alignment between the DNA sequences of the S-layer genes of C. difficile strains 1, 17 and 630. Nucleic acid residues of the DNA sequences in boxes are homologous in all three of the strains examined. Those not in boxes show no homology, or only homology between two out of three of the strains examined.

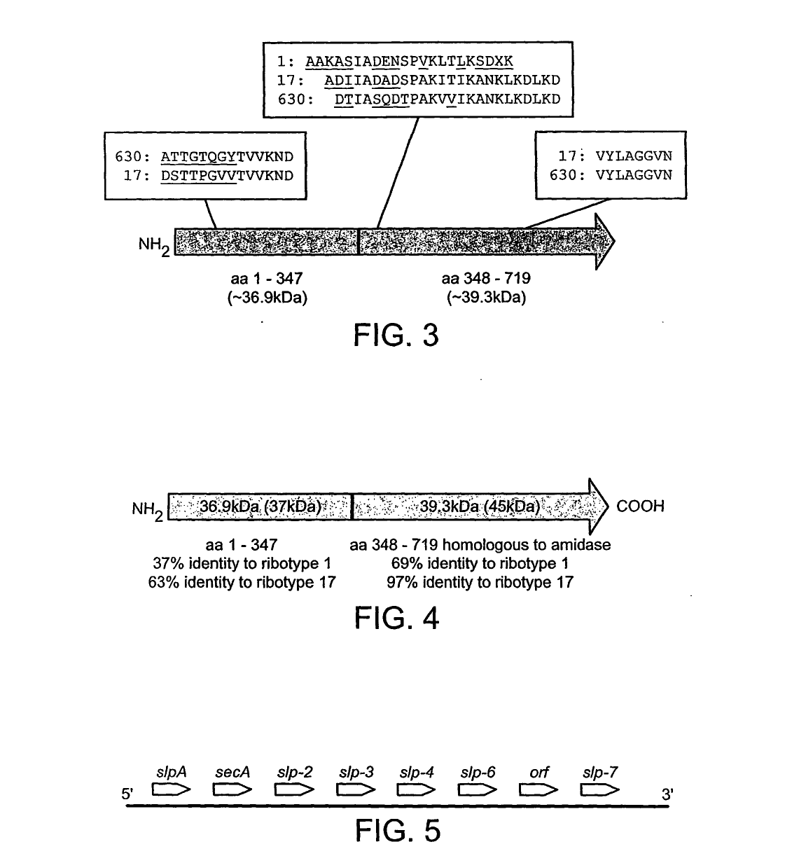

FIG. 3 shows the organisation of the S-layer genes in C. difficile. The boxes show selected regions of amino acid sequence from 3 strains, indicating the degree of amino acid conservation found. Underlined residues are not conserved. Residues 1-347 represent the “high MW” protein, whereas 348-719 represent the lower MW protein.

FIG. 4 shows the organisation of the S-layer gene (slpA) in C. difficile strain 630. The figures in kDa indicate the predicted MWs of the proteins; those in brackets are observed MWs by SDS-PAGE. The percentage amino acid identity to slpA from strains 1 and 17 are indicated below each domain.

FIG. 5 shows the arrangement of sip genes in C. difficile 630 “slp region 1”. The two domains encoded by the 5′ slpA gene are indicated in FIG. 4. DNA sequence predicts transcription of all genes from top strand of DNA.

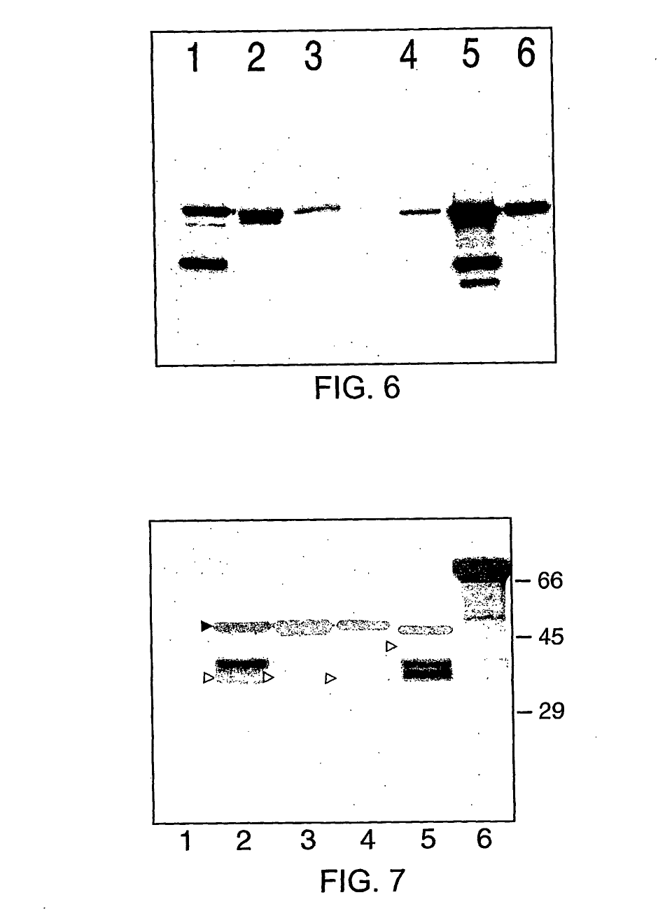

FIG. 6 shows the immunological cross reaction of SLPs from different C. difficile strains. Purified SLPs from strains 1 (lanes 1 and 4), 17 (lanes 2 and 5) and 630 (lanes 3 and 6) were reacted with antisera raised against SLPs from strain 1 (lanes 1,2,3) and strain 17 (lanes 4, 5, 6).

FIG. 7 shows glycan detection in SLP preparations from C. difficile. Lane 1, negative control (creatinase); lane 2, SLPs from strain 1; lane 3, SLPs from strain 17; lane 4, SLPs from strain 630; lane 5, SLPs from strain Y; lane 6, positive control (transferrin). The high molecular weight SLP is indicated by a solid arrow. The white arrow indicates the positions of the 33 kDa SLP in strains 1, 17 and 630 and the 38 kDa SLP in strain Y. The prominent bands of activity in lanes 2 and 5 are due to contaminating glycoproteins in the SLP preparations.

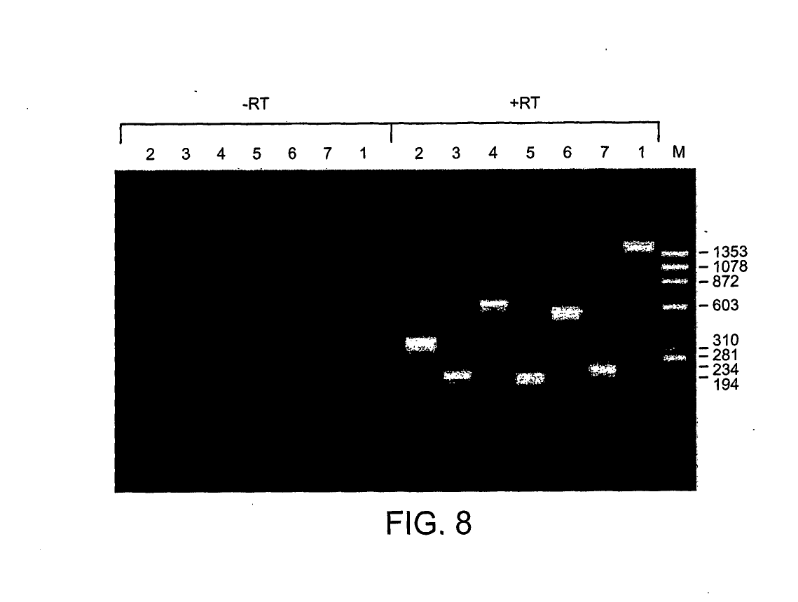

FIG. 8 shows RT-PCR of the ORFs downstream of slpA in C. difficile 630. RNA was isolated from a growing culture of C. difficile 630 and regions of each ORF were amplified by RT-PCR using primers specific for each gene. The size of the reaction products reflects the regions chosen for amplification within each sequence. Lanes 1-7, ORFs 1-7; +/−RT designates reactions carried out in the presence and absence of reverse transcriptase. M, molecular weight standards (bp).

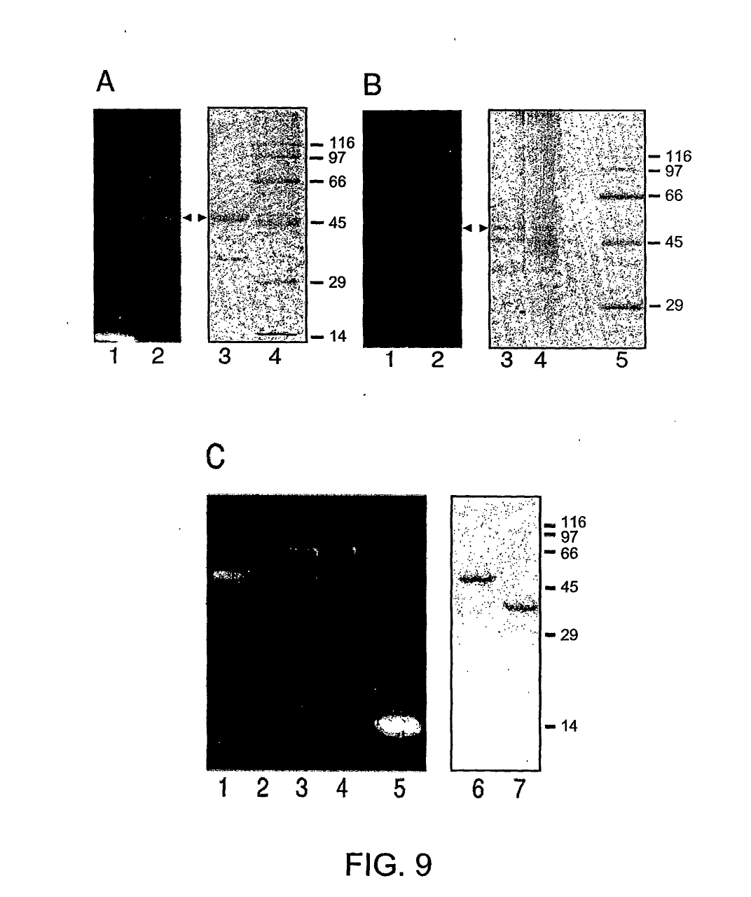

FIG. 9 shows the detection of amidase activity of the SLPs from C. difficile. FIG. 9A shows Zymogram (lanes 1 and 2) and Coomassie stain (lanes 3 and 4) of SLPs from C. difficile strain 17. Lanes 1 and 4, molecular weight standards; lanes 2 and 3 SLPs extracted from C. difficile. FIG. 9B shows Zymogram (lanes 1 and 2) and Coomassie stain (lanes 3-5) of cell extracts of C. difficile strain 17. Lanes 1 and 3, cytosolic fraction; lanes 2 and 4, membrane fraction, lane 5 molecular weight standards. The high MW SLP protein is arrowed. FIG. 9C shows Zymogram (lanes 1-5) and Coomassie blue stain (lanes 6 and 7) of recombinant and native SLPs. Lanes 1 and 6, high MW SLP purified from E. coli; lanes 2 and 7, low MW SLP purified from E. coli; lane 3, extracted SLPs from C. difficile strain 1, lane 4, extracted SLPs from C. difficile strain 630; lane 5, MW standards.

### EXAMPLES 1-10

### Example 1

### Preparation of Surface Layer Proteins from C. difficile strains ribotypes 1 and 17.

Two strains of C. difficile are used in this example (“1” and “17”). These strains differ in their ribotype, being the sequence of the 16S ribosomal RNA (as determined by sequencing the DNA encoding the RNA).

S-layer proteins from C. difficile strains of ribotype #1 and #17 (strains 1 and 17 respectively) are prepared from cells by a modification of the method of Dubreuil (Dubreuil, J. D. et al (1988) J. Bacteriol. 170(9):4165-73). Briefly the cells are grown in Brain Heart Infusion broth, the cells concentrated by centrifugation and ished twice in phosphate-buffered saline (pH 7.2). The cells pellet is resuspended in 0.2 m glycine hydrochloride buffer (pH 2.2) and stirred for 20-30 mins at room temperature. Whole cells are removed by centrifugation, and the supernatant neutralised by addition of NaOH.

The S-layer protein preparations are shown to each contain 2 prominent proteins of approximate molecular weights 45 kDa and 36 kDa, which corresponded to the two main proteins observed in cell wall preparations from other strains of C. difficile. Because of their relative mobility on SDS-polyacrylamide gels, these proteins are referred to hereinafter as “upper band” (45 kDa) and “lower band” (36 kDa) respectively.

### Example 2

### Amino Acid Sequencing of the S-Layer Proteins

A. N-terminal Amino Acid Sequences

The N-terminal amino acid sequence of the “upper band” and lower band from strain ribotype #1 and ribotype #17 are obtained the N-terminal amino acid sequence of each protein from each strain is determined by Edman degradation.

Proteins are separated on a SDS-polyacrylamide gel and transferred to a PVDF membrane by electrotransfer. Individual bands corresponding to the upper band and lower band are excised from the PVDF membrane and subjected to gas phase sequencing. The results obtained are given below:

| | |
|---|---|
| #1 upper: | AAKASIADENSPVKLTLKSDXKX |
| #17 upper: | ADIIADADSPAKITIKANKLKDLKD(C)VDDL |
| #1 lower: | DDTKVETGDQGYTVVQSKKYKAAVEQLQKI |
| #17 lower: | DSTTPGVVTVVKND |

where
X = uncertain amino acid
(C) = possibly cysteine but uncertain

where

X=uncertain amino acid

(C)=possibly cysteine but uncertain

B. Internal amino acid sequences

The two S-layer protein samples derivable from strains 1 and 17 are initially analysed using a SDS-PAGE electrophoresis (10%) on a BioRad Mini-PROTEAN II system. Each of these samples separates to give two distinct bands on coomassie staining. The two bands (derived from each strain), at approximately 35 and 40 kD respectively, are excised and exposed to tryptic in-gel digestion.

Each band is first excised from the gel, cut into small pieces and then destained by incubating in approximately 200 ul 60% Acetonitrile:100 mM Ambic solution for approximately 10 minutes at R.T. After removal of the added solvent, the gel is freeze dried and lug of Porcine Trypsin in 20 ul 100 mM Ambic (pH 8.4) is added. Once the enzyme solution had been absorbed, Ambic is added to cover the gel pieces and the sample is then incubated overnight at 37° C.

After digestion, the peptides are extracted from the gel by first removing the Ambic in the sample for pooling. The gel pieces are then covered with 0.1% TFA and incubated for two hours to stop the digestion, after which the 0.1% TFA is removed and pooled with the Ambic. Subsequently, the gel pieces are covered once more with 60% acetonitrile in 0.1% TFA for 2 hours, after which the solvent is removed and pooled with the Ambic/0.1% TFA. This 60% Acetonitrile in 0.1% TFA step is repeated three times using half hour incubations with pooling after each step. The pooled sample extracts containing the peptides are then concentrated to a volume of approximately 10-20 ul ready for purification.

The concentrated sample is purified by first loading onto a 1×10 mm C18 reverse phase cartridge (Jones Chromatography) using the Applied Biosystem microbore HPLC system operated at a flow rate of 10 ul/min using 0.1% TFA. The cartridge is then ished for 15 minutes using 0.1% TFA and the peptides are eluted isocratically using a 30% acetonitrile/0.1% TFA solution. Fractions are collected every 30 seconds. 1-2 ul of the fraction collected at the UV peak top is loaded into a metal-coated glass capillary for analysis by nanospray Q-TOF MS and MS/MS.

Application of this procedure results in 2 peptide sequences from each protein; thus eight peptide sequences are generated in total. The peptides sequences elucidated are as follows:

Ribotype #1

| | |
|---|---|
| 1 upper | YYNSDDENA |
| 1 upper | VGGTGL/IADAM |
| 1 lower | YQVVI/LY |
| 1 lower | VGSEL/INAAD |

Ribotype #17

| | |
|---|---|
| 17 upper | VDAL/IAAA |
| 17 upper | VYL/IAGGVN |
| 17 lower | YQVL/IFY |
| 17 lower | TVDTASNEAFAGDGK |

where I/L indicates Leucine or Isoleucine

### Example 3

### Analysis of Genome Sequence of C. difficile Strain 630

The putative S-layer genes of C. difficile strain 630 are identified using the sequence similarity search tool (BLAST) using freely available software, e.g. located at http://www.ncbi.nlm.nih.gov/BLAST/. Briefly the amino acid sequences obtained experimentally are used as probes to search the data generated by the C. difficile genome sequencing project (www.sanger.ac.uk/Projects/C_difficile/.)

The genes encoding the S-layer proteins from ribotypes 1 and 17 are cloned by PCR amplification using oligonucleotides derived from the genes for the S-layers proteins from strain 630. Chromosomal DNA from strains of ribotypes 1 and 17 are prepared by the method of Wren (1987) with modifications as follows. Strains are grown in 50 ml Brain Heart Infusion broth for 48 hours, the cells centrifuged and the pellet resuspended in 3 ml IM sucrose. 3 ml of buffer A (50 mM Tris-HCl (pH 8.0), 50 mM EDTA) is added followed by 0.6 ml 10 mg/ml lysozyme in buffer B (10 mM Tris-HCl pH 8.0, 10 mM EDTA). After incubation at 37° C. for 30 minutes, 350 ul 10% SDS and 100 ul Proteinase K (20 mg/ml in water) are added and incubation continued at 50° C. for 1 hour. After cooling to room temperature, 350 ul 5M NaCl and 5 ml isopropanol are added and the solution mixed gently until a precipitate of DNA appears. The precipitate is transferred to a microfuge tube containing 0.5 ml 70% ethanol, mixed gently and centrifuged for 30 seconds in microfuge. The supernatant is removed and the pellet allowed to dry. The pellet is resuspended in 0.4 ml TE (10 mM Tris-HCl pH 8.0, 1 mM EDTA). 4 ul RNase 10 mg/ml is added and the solution is incubated at 37° C. for 30 min. 20 ul 5M NaCl is added followed by 0.4 ml phenol:chloroform (1:1) and the solution centrifuged for 5 min in microfuge. The aqueous phase is precipitated with ethanol and washed with 70% ethanol and resuspended in a suitable volume of TE buffer (10 mM Tris-HCl pH 8.0, 1 mM EDTA).

PCR amplification is carried out by standard methods. DNA fragments are gel purified and cloned into E. coli plasmid vectors using standard molecular biology techniques, for example as described by Sambrook et al., (Molecular Cloning, A Laboratory Manual (1989)). DNA sequence is determined by standard methods.

This method analyses the DNA sequence of strain 630 for regions which, when translated, show significant homology to the peptide sequences obtained for ribotypes 1 and 17.

The analysis shows that one open reading frame in 630 contains significant homology to peptides from both the upper and lower bands from both ribotype strains 1 and 17. However, the homology is not complete and significant differences are also apparent. Examples of this heterogeneity are shown in the Figures.

### Example 4

### Comparisons of Protein and DNA Sequences of the S-Layer Proteins from C. Difficile Strains 630 and Ribotypes 1 and 17

The DNA sequences of the S-layer genes of C. difficile strains 1, 17 and 630 are determined, and the amino acid sequences deduced. The DNA sequences from the 3 strains are aligned and are shown in FIG. 2. The amino acid sequences are aligned and are shown in FIG. 1.

The examples (including the alignments of both DNA and proteins sequences) show the following:

i) two S-layer proteins (36 and 45 kDa) appear to be processed from a common large precursor protein, and are not two separate genes as expected.

(ii) a “classical” predicted signal sequence at the N-terminus of the ORF.

iii) little sequence homology between the 45 kDa and 36 kDa proteins from individual strains, suggesting the proteins may have distinct functions.

iv) the 36 kDa proteins from the 3 strains exhibit lower amino acid sequence homology to each other than to the 45 kDa proteins

v) database BLAST searches reveal the 45 kDa proteins exhibit homology to N-acetyl muramoyl L-alanine amidase (amidase), a peptidoglycan hydrolase which catalyses the cleavage of L-alanine from muramic acid present in the peptidoglycan.

vi) the SLH (Surface Layer Homology) motif is absent from all strains. This complex amino acid motif (see www.sanger.ac.uk/Software/Pfam/) is found in some, but not all bacterial S-layer proteins.

vii) the (36 kDa) corresponds to the N-terminal processed product. The higher MW protein is estimated by SDS-PAGE to be −45 kDa, significantly higher than the 39.3 kDa predicted from the sequence. This size difference is probably in part due to glycosylation as demonstrated by analysis of the S-layer proteins by mass spectrometry.

### Example 5

### Analysis of the Strain 630 Genome Sequence

Analysis of the strain 630 genome sequence reveals several genes downstream of the slpA gene which encode proteins with partial identity (33%-51%) to amidase. These proteins are all transcribed in the same direction as slpA and may be part of an operon. Interestingly the genes are not all contiguous, some being separated by other genes, for example the seca gene. Hereinafter this region is referred to as “sip region 1.”

Using RT-PCR the present inventors show, perhaps surprisingly, that all slp genes in this region are transcribed. Further sequence analysis of the 630 genome sequence reveals the presence of over 20 more genes, all with homology (25-30% identity) to the amidase from either C. difficile or B. subtilis.

### Example 6

### The molecular Basis for Diversity in S-Layer Expression in C. difficile

This example explores the molecular basis for the observed variation in SLPs expression in C. difficile observed by SDS-PAGE (16,19,21). The SLP genes from strains of C. difficile are cloned which express different sized SLPs. Strains are available from Dr John Brazier, Anaerobe Reference Centre, Cardiff and from Professor Peter Borriello, Public Health Labs, Colindale. Possible reasons for different sized SLPs in these strains are: (1) alternative processing of slpA at distinct sites to yield proteins of different sizes; (2) distinct sip genes expressed in strains which exhibit different patterns of SLPs, perhaps a homolog of those identified by our genome analysis; (3) the slpA gene in these strains contains insertions or deletions; (4) alternative degrees of glycosylation of slpA. Chromosomal DNA is prepared from relevant strains and the sip genes cloned either by constructing genomic banks in λGEM11 or by using PCR with Pfu polymerase to reduce errors, initially using oligonucleotides specific to regions of slpA which are conserved between strains 1, 17 and 630 (to avoid amplification of other slp genes). In the event that the slpA in these strains is not homologous enough to clone by PCR, oligonucleotides based on secA are used, downstream from slpA in 630 and ribotype 1. slpA genes from several strains are then be completely sequenced. Antibodies are raised against the purified SLPs from representative strains. These are used to detect immunological relatedness of SLP from a range of strains. It is thus possible to identify defined regions or domains within these proteins and to develop defined sera to type C. difficile and aid in the diagnosis of infection.

FIG. 6 shows the immunological relatedness of the SLPs from C. difficile strains. Here, immunological cross reaction of SLPs from different C. difficile strains. Purified SLPs from strains 1 (lanes 1 and 4), 17 (lanes 2 and 5) and 630 (lanes 3 and 6) were reacted with antisera raised against SLPs from strain 1 (lanes 1,2,3) and strain 17 (lanes 4, 5, 6).

### Example 7

### Characterisation of the S-Layer Proteins from C. difficile Strain 630

The SLPs from strain 630 are purified from overnight cultures by low pH extraction, and the amino acid sequence of peptides within both bands is determined by mass spectrometry. This establishes whether the SLPs are expressed from the slpA gene. The divergence of sequence at the N-termini of SLPs allows the unambiguous determination of which sip gene is translated to yield the SLPs. If, like strains of ribotype 1 and 17, the slpA homolog constitutes the SLP, this suggests that C. difficile has one primary gene which expresses S-layer proteins. If the amino acid sequence does not correlate with slpA, BLAST searches of the genome reveals which gene is expressed in strain 630.

It is thought that the SLPs from C. difficile are glycosylated. Experiments may be performed to establish the glycosylation status of both SLPs from strain 630. Individual SLPs, purified by ion exchange chromatography, are digested with trypsin and fragments analysed by mass spectrometry using QTOF. Assuming glycosylation is evident from this analysis, further experiments are performed to analyse the sugar content of the proteins after hydrolysis. This analysis is repeated with SLPs from other strains to complement the sequence analysis. Proteins may be purified further by ion exchange chromatography (Takeoka, A., et al (1991) J Gen Microbiol 137(Pt 2), 261-7) prior to analysis. Individual SLPs, produced by expression in E. coli, are also analysed to assess whether these proteins can be glycosylated in heterologous species.

FIG. 7 shows the glycosylation of the SLPs. Here, glycan detection in SLP preparations from C. difficile. Lane 1, negative control (creatinase); lane 2, SLPs from strain 1; lane 3, SLPs from strain 17; lane 4, SLPs from strain 630; lane 5, SLPs from strain Y; lane 6, positive control (transferrin). The high molecular weight SLP is indicated by a solid arrow. The white arrow indicates the positions of the 33 kDa SLP in strains 1, 17 and 630 and the 38 kDa SLP in strain Y. The prominent bands of activity in lanes 2 and 5 are due to contaminating glycoproteins in the SLP preparations.

### Example 8

### Transcriptional Studies

Transcriptional analysis is performed of the “slp region 1” in strain 630. slpA and all downstream genes are transcribed in the same direction as slpA, (FIG. 5) suggesting the possibility of polycistronic transcript(s). The length of the slpA transcript is determined and the presence of read-through transcripts into seca or the other downstream genes is investigated. Previous analysis by RT-PCR demonstrates that there is sufficient DNA sequence diversity to design specific PCR primers for each putative slp gene. The same analysis may be performed on the >20 amidase homologs present in strain 630. The DNA upstream of slpA is analysed for the presence of any putative regulatory genes to analyse the mechanisms of control of SLPs in C. difficile. The transcriptional start site of sipa is determined by primer extension as described for the C. difficile toxin AB locus (Hundsberger, T. et al (1997) Eur J. Biochem 244(3), 735-42).

FIG. 8 shows some results of the transcriptional studies. Here, RT-PCR of the ORFs downstream of slpA in C. difficile 630. RNA was isolated from a growing culture of C. difficile 630 and regions of each ORF were amplified by RT-PCR using primers specific for each gene. The size of the reaction products reflects the regions chosen for amplification within each sequence. Lanes 1-7, ORFs 1-7 (see FIG. 4); +/−RT designates reactions carried out in the presence and absence of reverse transcriptase. M, molecular weight standards (bp).

### Example 9

### Investigation of Putative Enzyme Function of the S-Layer Proteins.

BLAST searches reveal homology of the C-terminal domain of slpA to N-acetyl muramoyl L-alanine amidase (amidase), an enzyme essential for peptidoglycan biosynthesis and turnover. Many ORFs in bacteria whose genome sequence have been determined have been annotated as homologs of amidase (the genome sequence of B. subtilis contains 11 putative amidase homologs, (Kunst F. (1997) Nature 390: 249-256) but relatively little work on their expression and function has been carried out.

In this example, the C-terminal slpA domain is expressed in E. coli, either as a cytoplasmic protein using a 6×His tag vector (pET28) or within the periplasm using pMALc2. Amidase activity is assayed from both cloned proteins and from SLPs from C. difficile using an in-gel assay (Lantz M. S. and Ciborowsld P. (1994) Methods Enzymol 235 563-594) where proteins are separated on an SDS-PAGE gel containing C. difficile cell wall peptidoglycan substrate. Renaturation of the proteins allows enzyme activity to be revealed by decolourisation of methylene blue at the site of the protein band. The present inventors have established this method using lysozyme. The experiments may be repeated with other putative slp genes in strain 630 to determine if they encode active enzymes.

FIG. 9 shows the results regarding investigations into the function of the S-layer proteins, namely by the detection of amidase activity of the SLPs from C. difficile. With reference to FIG. 9:

A. Zymogran (lanes 1 and 2) and Coomassie stain (lanes 3 and 4) of SLPs from C. difficile strain 17. Lanes 1 and 4, molecular weight standards; lanes 2 and 3 SLPs extracted from C. difficile.

B. Zymogram (lanes 1 and 2) and Coomassie stain (lanes 3-5) of cell extracts of C. difficile strain 17. Lanes 1 and 3, cytosolic fraction; lanes 2 and 4, membrane fraction, lane 5 molecular weight standards. The high MW SLP protein is arrowed.

C. Zymogram (lanes 1-5) and Coomassie blue stain (lanes 6 and 7) of recombinant and native SLPs. Lanes 1 and 6, high MW SLP purified from E. coli; lanes 2 and 7, low MW SLP purified from E. coli; lane 3, extracted SLPs from C. difficile strain 1, lane 4, extracted SLPs from C. difficile strain 630; lane 5, MW standards.

### Example 10

### Analysis of S-Layer Protein Expression in Human Isolates of C. difficile.

Previous work has shown that human antibodies are generated against cell wall proteins of C. difficile during a natural infection (Pantosti (1988) J. Clin. Microbiol. 27(11), 2594-7) and that a 36 kDa SLP from C. difficile C253 is immunodominant (Cerquetti et al (1992) Microb Pathog. 13(4)271-9). However, the SLPs in these studies were not identified, characterised or sequenced.

In this example, a range of strains from patients with CDAD are collected, together with matched convalescent sera (available from Dr Lewis, Adenbrooks Cambridge). The specificity of the antibody response is investigated to the SLPs using cloned SLPs as antigens in ELISA and western blots. Extensive sequence identity is observed between the SLPs from 3 strains, particularly in the amidase domain, and suggests a degree of cross reactivity between the homologous proteins from strains. This example establishes whether an antibody response to one or more SLPs is important in convalescence from C. difficile infections.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

1. A polypeptide comprising the amino acid sequence shown in SEQ ID No. 1, SEQ ID No. 2 or SEQ ID No. 3, or a homologue, variant or derivative thereof.

2. A polynucleotide capable of encoding a polypeptide according to claim 1.

3. A polynucleotide according to claim 2, comprising the nucleic acid sequence shown in SEQ ID No. 4, SEQ ID No. 5 or SEQ ID No. 6, or a homologue, variant or derivative thereof.

4. A peptide comprising a portion of a polypeptide according to claim 1.

5. A peptide according to claim 4 which comprises one or more regions which are homologous between at least two of SEQ ID No. 1, SEQ ID No. 2 and SEQ ID No. 3.

6. A peptide according to claim 4 which comprises one or more regions which are heterologous between at least two of SEQ ID No. 1, SEQ ID No. 2 and SEQ ID No. 3.

7. A nucleotide capable of encoding a peptide according to any of claims 4 to 6.

8. A vector comprising a polynucleotide according to claim 2 or 3, or a nucleotide according to claim 7.

9. A host cell comprising a vector according to claim 8.

10. A method for screening for a compound which is capable of interacting specifically with a C. difficile S-layer protein, using the polypeptide of claim 1, or a peptide according to any of claims 4 to 6.

11. A compound capable of binding specifically to a polypeptide according to claim 1 and/or to a peptide according to any of claims 4 to 6.

12. The use of a polypeptide according to claim 1, or part thereof; a polynucleotide according to claim 2 or 3, or part thereof, a peptide according to any of claims 4 to 6; or a nucleotide according to claim 7, in a method for producing antibodies.

13. An antibody capable of binding specifically to a polypeptide according to claim 1 and/or to a peptide according to any of claims 4 to 6.

14. A pharmaceutical composition comprising: a polypeptide according to claim 1, or part thereof; a polynucleotide according to claim 2 or 3, or part thereof; a peptide according to any of claims 4 to 6; a nucleotide according to claim 7; a vector according to claim 8; or an antibody according to claim 13.

15. An immune modulating composition comprising a polypeptide according to claim 1, or part thereof; a polynucleotide according to claim 2 or 3, or part thereof; a peptide according to any of claims 4 to 6; a nucleotide according to claim 7; a vector according to claim 8; or an antibody according to claim 13.

16. A method for treating and/or preventing a disease in a subject, which comprises the step of administering: a polypeptide according to claim 1, or part thereof; a polynucleotide according to claim 2 or 3, or part thereof; a peptide according to any of claims 4 to 6; a nucleotide according to claim 7; a vector according to claim 8; an antibody according to claim 13; a pharmaceutical composition according to claim 14; or an immune modulating composition according to claim 15, to the subject.

17. A method according to claim 16, wherein the disease is associated with Clostridium difficile infection.
